(19) United States
(12) Patent Application Publication
(10) Pub. No.: US 2009/0131798 A1
(43) Pub. Date: May 21, 2009

(54) METHOD AND APPARATUS FOR INTRAVASCULAR IMAGING AND OCCLUSION CROSSING

(76) Inventors: Christopher D. Minar, New Prague, MN (US); Peter Skujins, Minneapolis, MN (US); Raju R. Viswanathan, St. Louis, MO (US); Guy Besson, St. Louis, MO (US); Gareth T. Munger, St. Louis, MO (US)

Correspondence Address: Bryan K. Wheelock, Suite 400, 7700 Bonhomme, St. Louis, MO, 63105, US

(21) Appl. No.: 12/273,593

(22) Filed: Nov. 19, 2008

Related U.S. Application Data
(60) Provisional application No. 61/019,231, filed on Jan. 4, 2008.
(60) Provisional application No. 60/989,112, filed on Nov. 19, 2007.

(51) Int. Cl. A61B 8/14; A61B 18/14; A61M 25/01

(52) U.S. Cl. 600/463; 600/471; 606/41; 604/528

(57) ABSTRACT

This invention describes an occlusion crossing apparatus for creating an opening in occluded tissue at a target location in interventional and surgical applications with simultaneous or nearly simultaneous intravascular imaging. In particular, the present invention is concerned with a magnetically guidable occlusion crossing apparatus and methods of using same together with intravascular ultrasound imaging, said occlusion crossing apparatus being usable in combination with a magnetic field and comprising an ultrasound imaging catheter with at least one ultrasound transducer at its distal tip; the catheter including a lumen through which a magnetically steered guidewire is passed and extends beyond the distal end of the catheter; the guidewire possibly comprising an electrode at its distal end for delivery of ablative electrical energy at a target location in a body lumen; and at least one magnetic guiding element mounted to the guidewire for orienting the portion of the guidewire that extends from the distal tip of the imaging catheter and placing the tip of the guidewire at the desired target location.

### CROSS-REFERENCE TO RELATED APPLICATIONS

This application claims priority to both U.S. Provisional Patent Application No. 61/019,231, filed Jan. 4, 2008 and U.S. Provisional Patent Application No. 60/989,112, filed Nov. 19, 2007, the entire disclosures of which are incorporated herein.

### FIELD

The present invention relates generally to methods and devices to cross occlusions in interventional and surgical applications with simultaneous or nearly simultaneous intravascular imaging, and in particular, to a magnetically guidable energy delivery or occlusion crossing apparatus and methods of using same together with intravascular ultrasound imaging.

### BACKGROUND

Many medical interventions rely on the delivery to a target location of energy, such as electrical energy, inside the body of a patient. For example, an occlusion in a blood vessel such as a partial or total occlusion may be vaporized, at least partially, by delivering a suitable electrical current to the occlusion.

There currently exist magnetically guided guidewires, which are typically relatively long and relatively thin wires at the end of which a magnet is located. The guidewire is typically used in conjunction with a catheter that is slid over the guidewire after the wire has been advanced through a desired path. In use, the guidewire protrudes a relatively small distance in front of the catheter when there is a need to either steer the catheter at a junction, or guide the catheter through a relatively tortuous path. Then, a magnetic field may be applied to guide the guidewire through a predetermined path. Thereafter, the catheter is slid over the guidewire. The guidewire can have an electrode at the tip that can be used to deliver RF energy at the tip for local ablation and removal of tissue. In the local vicinity of the tip of such a device, however, it is important to ensure that the wall of the blood vessel that is being ablated is not perforated, and that only the blockage within the vessel is ablated.

There is a need to provide novel remotely steerable devices that can not only be navigated efficiently and deliver energy effectively to or effectively push through occlusion at a desired lesion site in the patient anatomy, but can also provide local imaging that can help ensure that ablation is occurring safely away from vessel walls. The present invention is designed to provide such a method and an apparatus.

### SUMMARY

In a broad aspect, the invention provides an occlusion crossing apparatus in the form of an energy delivery apparatus for delivering electrical energy at a target location, the energy delivery apparatus being usable in combination with a magnetic field. The energy delivery apparatus can be in the form of a guidewire that acts as an electrical conductor or it can be in the form of a catheter that incorporates a lead that acts as an electrical conductor, in addition to having sufficient flexibility in its distal portion to be navigated efficiently through tortuous anatomy. An electrode at the wire or apparatus tip is used for delivering the electrical energy at the target location, the electrode being electrically coupled to the electrical conductor and located at a predetermined location therealong; also included is a guiding element mounted to the electrical conductor in a substantially spaced apart relationship relative to the electrode, the guiding element including a magnetically responsive material. The energy delivery apparatus is constructed such that a movement of the guiding element causes a corresponding movement of the electrode. An external magnetic field is applied to move the guiding element in order to position the electrode substantially adjacent to the target location.

In one embodiment, the invention provides an occlusion crossing apparatus in the form of a magnetically steered device for pushing through an occlusion at a target location, the occlusion crossing apparatus being usable in combination with a magnetic field.

Further included as part of the catheter apparatus is at least one ultrasonic transducer. The ultrasonic transducer can emit and receive ultrasonic energy and can comprise a piezoelectric element. When multiple transducers are used, they can be located in ring-like fashion around the circumference of the energy delivery apparatus. Each transducer has leads that pass through the energy delivery apparatus and connect to an ultrasound system through a suitable connector at the proximal end of the device. The latter can be a catheter of suitably small diameter. For non-specific purposes of illustration only, the device diameter can be in the range 2-6 French or 0.66-2 mm. The transducers could be used to image independently or in phased array form to acquire a circumferential imaging pattern and to view multiple directions near-simultaneously in real time. If the transducers are placed close enough to provide overlapping fields of view, a continuous ring-like annular field of view can be obtained.

The energy delivery apparatus in one embodiment can comprise the imaging catheter including the ultrasonic transducers together with an electrode at one location along the catheter circumference spaced away from the transducers, with the catheter incorporating magnetic elements that can be used to steer the device by application of an external magnetic field. When this electrode is used for ablation, the transducers can be used to provide an image in a circumferential wedge-like sector that is across from the electrode location. In this imaging view, if the vessel wall is visible close to the catheter, it indicates that the device electrode is relatively well centered and it is safe to ablate, as long as the device diameter is no more than about half the healthy vessel diameter.

The transducers could transmit an ultrasound beam directed radially away from the imaging catheter, in which case, the device is a side-viewing catheter, or it could transmit an ultrasound beam angled away from the axis of the device at an angle less than 90 degrees, in which case, the image obtained is an oblique view and the catheter is an oblique-viewing device.

In another embodiment, the imaging catheter is distinct from the energy delivery apparatus, but contains a passage through which the energy delivery apparatus in the form of a magnetic RF guidewire is passed. The RF guidewire has an electrode for RF energy delivery to tissue for creation of an opening through a lesion. The channel or passage in the imaging catheter through which the RF wire passes can be centrally located in one embodiment, or it can be eccentrically located in another embodiment with respect to the long axis of the imaging catheter. When the ultrasound image shows that the vessel wall is close to the imaging catheter, the magnetic RF guidewire can be suitably steered in a direction slightly away from the vessel wall for ablation purposes at the wire tip by application of a suitable external magnetic field.

When only a single ultrasound transducer is used, the ultrasound image obtained from the catheter is in the form of a somewhat narrow circular sector. In this case, the catheter can be rotated or torqued about its long axis to obtain images from several such sectors. Such rotation of the device can be performed manually or it can be done under remote control by the use of at least one motor and a suitable drive mechanism which mechanically grips the device.

In some embodiments of the invention, when the catheter is directly used to ablate, a heat shield is located between the electrode and the transducers and guiding element(s) to prevent excessive heating of the latter two components. This improves the thermal insulation between the components and therefore further prevents de-magnetization of the magnetically responsive material present in the guiding element(s) and damage to the transducers. In another embodiment, where the imaging catheter is used together with an RF wire, the tip of the catheter is built from heat resistant material to ensure adequate thermal separation of the catheter's ultrasonic transducers from the RF guidewire electrode tip.

Other objects, advantages and features of the present invention will become more apparent upon reading of the following non-restrictive description of certain embodiments thereof, given by way of example only with reference to the accompanying drawings.

### BRIEF DESCRIPTION OF THE DRAWINGS

FIG. 1A is a side view of an ultrasound imaging catheter with a single side-viewing ultrasonic transducer in accordance with an embodiment of the present invention;

FIG. 1B is a side view of an ultrasound imaging catheter with a single oblique-viewing ultrasonic transducer in accordance with an embodiment of the present invention that provides an image looking forward and sideways at the same time;

FIG. 2 is a side view of an embodiment of an ultrasound imaging device in accordance with the principles of the present invention driven through a remotely operated advancer/rotation mechanism for remote operation of the device;

FIG. 3 is a side view of an embodiment of an imaging catheter with multiple ultrasonic transducers that can be used in phased-array fashion, together with a magnetic RF guidewire;

FIG. 4 is a side view of an embodiment of an ultrasonic imaging catheter with piezoelectric thin film transducers helically wound near the tip of the catheter;

FIG. 5 is a side view of an embodiment of a guide catheter that can accommodate an ultrasound imaging wire together with a magnetic RF guidewire, the latter devices for use in alternating fashion to cross a Chronic Total Occlusion;

FIG. 6 is a side view of an embodiment of a magnetically steered energy delivery apparatus in the form of an imaging catheter with an ultrasonic transducer at the distal tip, an electrode for delivery of RF energy positioned circumferentially opposite the transducer, and a magnetic element for steering the energy delivery apparatus;

FIG. 7 is an end view of an embodiment of a magnetically steered energy delivery apparatus in the form of an imaging catheter with a set of multiple ultrasonic transducers at the distal tip, and a set of multiple electrodes for delivery of RF energy positioned circumferentially opposite the set of transducers;

FIG. 7A is an end view of an alternate embodiment of a magnetically steered energy delivery system with a set of multiple ultrasonic transducers and multiple electrodes;

FIG. 8A is a perspective view of the distal end of a steered energy delivery device with a micro-electromechanical structure (MEMS);

FIG. 8B is a perspective view of the distal end of a steered energy delivery device; and

FIG. 8C is a partial side elevation view of the distal end of a steered energy delivery device.

### DETAILED DESCRIPTION

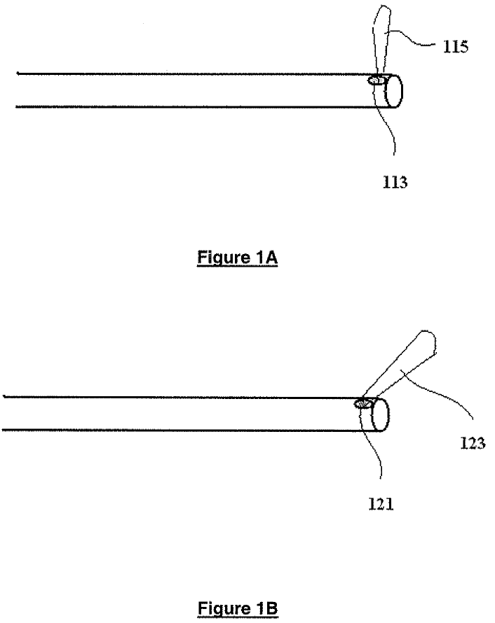

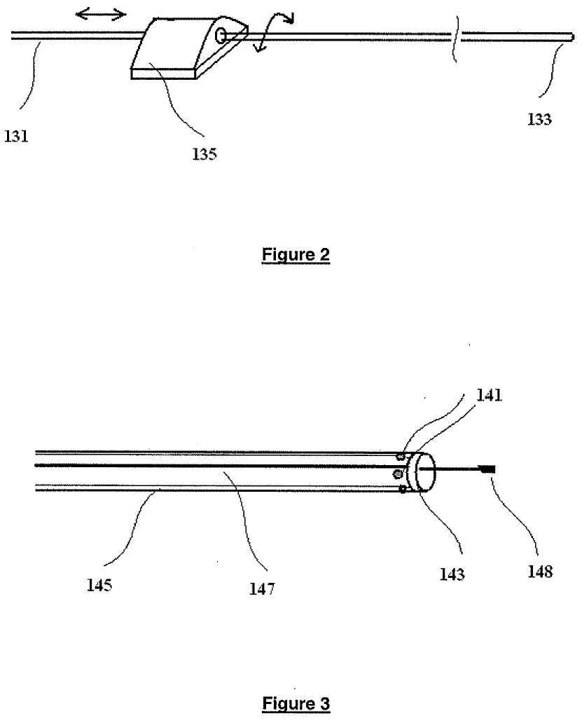

In one embodiment of the invention as shown in FIG. 1A, the imaging catheter has an ultrasonic transducer 113 mounted at the distal tip, capable of imaging radially outward in a circular sector 115. The catheter has a lumen for passage of a magnetically guided RF guidewire that can be used to enlarge a partially or completely occluded blood vessel. Based on the image seen in the field-of-view sector, a vessel wall seen close to the surface of the imaging catheter indicates that the guidewire is to be steered away from the wall in order to keep a safe distance away from the wall while ablating; the magnetic navigation system that controls the steering of the guidewire can be suitably operated to apply an appropriate magnetic field for this purpose.

In an alternate embodiment depicted in FIG. 1B, the ultrasonic transducer 121 produces an oblique field of view 123 with both forward and lateral components. The resulting image data, obtained in the form of tissue density as a function of oblique distance, can be displayed either directly as a partial sector-of-a-cone, or it can be warped or mapped into an annular sector where the radial coordinate is really an oblique measure of distance. One way to compute such a map is to project the oblique sector axially onto a plane representing the base of an appropriate cone reflecting the oblique angle, so that each point in the annular sector image has a radial coordinate that faithfully represents radial distance, while at the same time displaying a radially aligned distance scale with distance markings proportional to (cos α), where α is the cone half-angle defined by the transducer beam. These distance markings then indicate the forward distance in front of the catheter corresponding to a given radial location in the image. The catheter has a lumen for passage of a magnetically guided RF guidewire that can be used to enlarge a partially or completely occluded blood vessel. Based on the image seen in the field-of-view sector, a vessel wall seen close to the surface of the imaging catheter indicates that the guidewire is to be steered away from the wall in order to keep a safe distance away from the wall while ablating; the magnetic navigation system that controls the steering of the guidewire can be suitably operated to apply an appropriate magnetic field for this purpose.

With a single ultrasonic transducer, the image produced at a time is in the form of a sector. The imaging catheter can be rotated or torqued about its axis so that several sectors of data are obtained for a more complete circumferential view of the vessel interior. In one mode of image acquisition with such a system, the ultrasound imaging system can acquire multiple sectors in sequence and display them in integrated form in a single circular display to image most of the interior of the vessel out from the catheter. The rotation of the device can be manually performed by the user/physician, or as shown in FIG. 2, the imaging catheter 131 can pass through a device translation/rotation apparatus 135 that can rotate the device about its axis so that the device distal tip 133 can be rotated a full 360 degrees as required for a complete circumferential view of the vessel interior. The device translation/rotation apparatus 135 is equipped with a suitable rotary sleeve for engaging the device and incorporates a motor or motor cable-driven mechanism for applying suitable torques. The apparatus 135 can also be used to translate or advance or retract the device during the course of the medical procedure and can be operated remotely from a control room by the user, or it could also be programmatically driven by computer control. Clearly, such translation/rotation can be implemented with either a side-viewing imaging catheter or with an oblique-viewing imaging catheter.

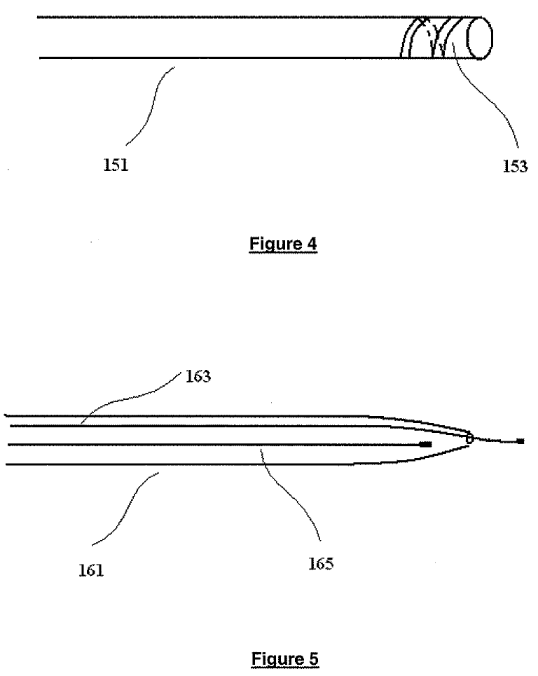

FIG. 3 illustrates one device embodiment where the imaging catheter comprises multiple ultrasonic transducers 141 deployed around the circumference of the imaging catheter. A heat shield 143 serves to separate the transducers from the RF electrode tip 148 of a magnetically steered RF guidewire 147 that passes through a lumen in the imaging catheter, so that as the RF guidewire is ablating through a vessel blockage the transducers are not harmed by excessive temperature increases. The imaging catheter can incorporate a metallic braiding 145 in its wall for enhanced torque transmission and/or to act as an electrical shield to prevent or minimize capacitive coupling between the RF guidewire 147 and external tissue around the imaging catheter. The incorporation of multiple transducers is useful for image acquisition in phased array form where image data can be obtained in overlapping sectors and combined by the ultrasound imaging system into a single circular display to provide a view of the entire vessel interior. If only a few transducers are present, the device can be rotated suitably about its axis in order to fill in any sector gaps in the image. The availability of real-time ultrasound image data is useful to determine potential steering directions for guidance of the magnetic RF guidewire so that the ablation is always safely performed well within the vessel interior and away from the vessel wall to avoid any vessel perforation risk.

In an alternate embodiment, the magnetically steered guidewire used with the ultrasound imaging catheter is not a Radio Frequency guidewire but rather a guidewire that is capable of mechanically pushing through a lesion. Such a guidewire, for instance, can comprise at least one guiding element constructed from a magnetically responsive material such as Neodymium-Iron-Boron, Platinum-Cobalt alloy, or other ferromagnetic or paramagnetic material. More than one guiding element can be used, for example a combination of a Neodymium-iron-Boron permanent magnet and a magnetized flexible coil built from Platinum-Cobalt alloy. For example, the latter combination can yield a magnetically steered guidewire that is both easily steered magnetically and can support a relatively large mechanical push force for crossing through an occlusion.

FIG. 4 shows one embodiment of an ultrasound imaging catheter for use with a magnetically guided RF wire for RF ablation energy delivery where the catheter incorporates ultrasonic transducers 153 in the form of piezo film transducers such as Kynar piezo film, helically wound in the distal portion of the catheter 151. Such a piezo film can provide a good acoustic impedance match to water/tissue for efficient transmission of ultrasound energy, is typically relatively low in cost, and is flexible and easy to form. While in some cases the total ultrasound energy output of such a thin film transducer may not be as high as that from piezo crystal transducers, it can still provide enough reflection data to produce a coarse image of the vessel interior that clearly delineates atherosclerotic plaque within the blood vessel.

One embodiment of the apparatus of the present invention is shown in FIG. 5. This figure shows a guide catheter 161 with a tapered distal tip section. The guide catheter carries two devices in its interior, a magnetically guided RF guidewire 165 and an ultrasonic imaging wire or microcatheter 163, shown with its distal tip (incorporating an ultrasound transducer) extending out of the guide catheter. The tapered distal section of the guide catheter permits extension of either the RF guidewire or the ultrasound wire from the distal tip. As an example of a method of use of this apparatus, the guide catheter is initially positioned at the proximal cap of a chronic total occlusion. Then the ultrasound wire is extended to provide an intravascular image and to assess the disposition of the vessel wall (the media/adventitia boundary) near the region of the guide catheter tip. A magnetic field is applied to suitably steer the magnetic RF guidewire so that it will tend to be well-centered with respect to the vessel, the ultrasound wire is withdrawn, and the magnetic RF guidewire is advanced. RF ablation energy is applied to ablate the tissue in front of the RF guidewire, and it is then advanced slightly through the opening created by the ablation. Then the RF guidewire is withdrawn, and the ultrasound wire is advanced into the opening in the lesion for further imaging data. This process is repeated in alternating fashion between the ultrasound wire and the magnetic RF guidewire, with the RF guidewire always being oriented by the magnetic field of the magnetic navigation system so as to remain well-centered within the vessel and kept away from the vessel wall.

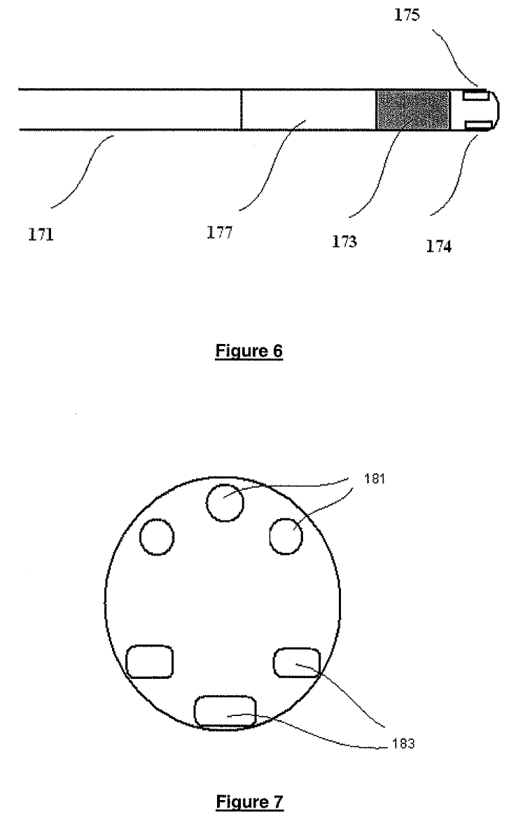

In one embodiment of the energy delivery apparatus of the present invention as shown schematically in FIG. 6, the energy delivery apparatus 171 performs the imaging function and the RF ablation function as a single integrated device. Thus, the distal tip of the device includes a RF ablation electrode 174 as well as an ultrasound transducer 175 situated circumferentially opposite the RF electrode. The tip region also includes at least one magnetic element 173 that responds to an external magnetic field causing the device to be steered into approximate alignment with the magnetic field. Just proximal to the magnetic element 173, the shaft 177 of the device is constructed from a suitably mechanically soft material and has a geometry such that the shaft 177 possesses a low bending stiffness to aid the magnetic steering of the device. In this embodiment, the same magnetically steered device is used for ultrasound imaging and for RF ablation, after being steered suitably in a direction away from a vessel wall based on the obtained intravascular imaging information. As described earlier, the energy delivery apparatus can be rotated either directly by a user, or in another embodiment, by a remote translation/rotation mechanism through which the energy delivery device passes.

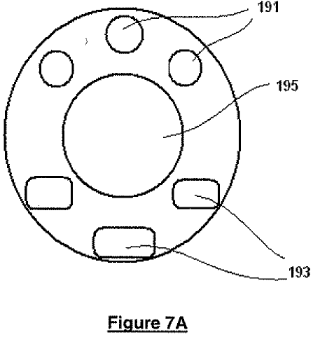

FIG. 7 shows another embodiment of a magnetically steered energy delivery apparatus in the form of an imaging catheter with a set of multiple ultrasonic transducers 181 at the distal tip, and a set of multiple electrodes 183 for delivery of RF energy positioned circumferentially opposite the set of transducers. With this arrangement, a wider sector field of view can be obtained together with ablation of a potentially wider cross section of tissue in the occlusion.

FIG. 7A shows another embodiment of an energy delivery apparatus in the form of an imaging catheter with a set of multiple ultrasonic transducers 191 at the distal tip, and a set of multiple electrodes 193 for delivery of RF energy positioned circumferentially opposite the set of transducers. The imaging catheter also has at least one through-lumen 195 through which another device such as a guidewire can be passed. In one preferred embodiment, the imaging catheter incorporates at least one magnetic element for magnetic steering. In one preferred embodiment, a guidewire passing through the lumen 195 incorporates at least one magnetic element for magnetic steering. The guidewire in a preferred embodiment includes an electrode at its distal tip for RF ablation. In one method of use of the imaging catheter, the electrodes on the imaging catheter are used for de-bulking or enlarging the lumen of an at least partially occluded vessel. An example of such usage is de-bulking of peripheral arteries in the treatment of patients with occluded vessels in the leg. Plaque or other deposits that clog a blood vessel and restrict normal blood flow can thereby be cleared away by RF ablation. The imaging catheter can be rotated about its long axis to ensure even de-bulking around the internal circumference of the vessel lumen, while at the same time the imaging derived from the catheter would help ensure that the actual vessel wall is not being breached or perforated. In one preferred embodiment of method of use, the guidewire that is passed through the imaging catheter can be used for creating an initial opening or pathway through an at least partially occluded vessel. The guidewire can be followed by a balloon or stent delivery catheter that is used to mechanically slightly enlarge the opening, followed by angioplasty balloon or stent delivery therapy to further open the vessel lumen to a near-normal internal diameter. Alternatively or in combination, RF ablation with the electrodes of the imaging catheter can be used to enlarge the opening of a vessel pathway initially created by RF ablation with a guidewire passed through the lumen of the imaging catheter. It is to be understood that either the imaging catheter, or the guidewire device passing through it, or both, can be steered by remote navigation means such as magnetic navigation. Variations of devices as described here and variations of methods of use or combinations of remote navigation and manual navigation means of the interior (such as guidewire) and exterior (such as imaging catheter) devices would be apparent to those skilled in the art. Accordingly the description given here is for illustrative purposes only without limitations on such variations.

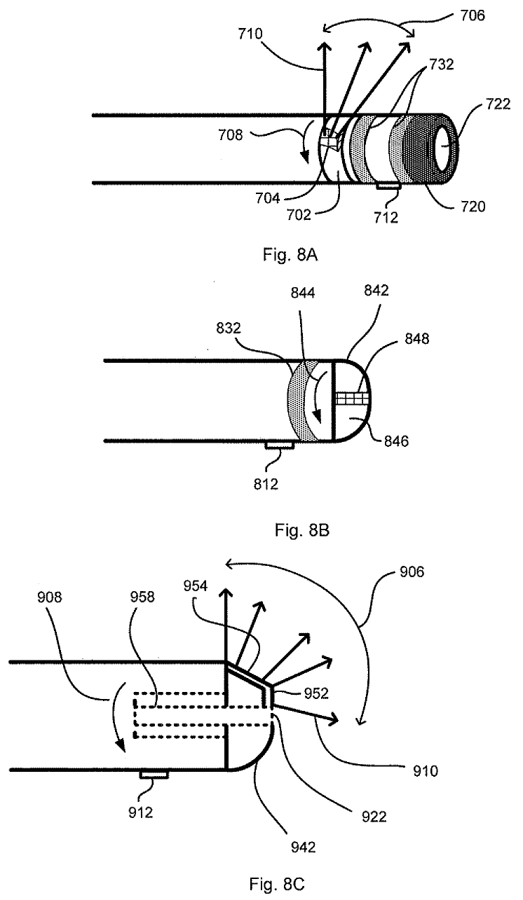

In another embodiment illustrated schematically in FIG. 8A, a catheter designed according to the principles of the present invention comprises a micro-electromechanical structure (MEMS) annulus or ring 702 located at or near the device distal tip. Mounted on the MEMS, ring is a small ultrasound transducer array 704 capable of sending and receiving ultrasound waves within an angular range 706 appropriate for body lumen imaging. The rotating annulus comprising a micro-slip ring structures allows rotation 708 around the device over an unlimited angle. The ultrasound array is preferably mounted on a forward angled surface within the rotating ring, such that ultrasound wave information 710 is acquired over a forward looking slice; as the ring is rotated, and depending on the radial distance from the catheter axis to a target site of ablation, the ultrasound array can image the target site, before, after, and possibly during application of the ablative power to RF electrode 712. The device distal end also comprises a hollow tip magnet 720. A guidewire or other medical device may be advanced within the catheter, through aperture 722, and beyond the distal tip into a lumen of interest. The distal tip configuration further comprises two insulating bands 732 of sufficient width and depth to thermally insulate both ultrasound array 704 and tip magnet from the heat generated by electrode 712 during ablation.

In an alternate embodiment, and as illustrated in FIG. 8B in a top view, and in FIG. 8C in a side view, the ultrasound probe is located on a semi-spherical device tip or ‘cap’ 842 that is rotatable 844 with respect to the long device axis. In one variation of this embodiment, a planar or curved surface 846 has been cut across the semi-spherical tip, allowing placement of an ultrasound transducer array 848 on one or more surface(s) 850, 852 at an angle to the axis, as also seen in a device side view presented in FIG. 8C. In this geometry, and depending on the design parameters, upon rotating the ultrasound probe can provide ultrasound image data extending over an angular range 906 sufficient to cover both the lumen wall, as well as the lumen axis. As in the previous embodiment, an RF electrode 912 is provided on the side of the device distal tip. Also provided are insulating band 932, as well as a tip magnet (not shown). In use, and after having been advanced within the patient to a location proximate to the lesion of interest, the ultrasound ‘cap’ rotates continuously 908 with respect to the device long axis 958, sending data through a slip-ring and data cables (not shown) or through the slip ring and to the system computer via an RF communication connection (not shown). The 3-dimensional (3-D) data thus acquired, enables precise localization of the target lesion; the device is then further advanced by a specified amount to put the RF electrode in contact with the lesion, and ablative power is applied. If necessary, the catheter is slowly retracted to enable imaging of the treated lesion. Control of the therapy may thus be made practical in quasi-real time. This process can be automated given the precise device control available through magnetic device navigation. As in the previous embodiment, a guidewire, therapy wire, or other medical device may be advanced within the catheter and through aperture 922 beyond the catheter distal end.

The magnetic RF guidewire in some embodiments of this invention includes an electrically insulating material substantially covering the electrically conducting wire shaft and made of a dielectric material with a relative dielectric constant preferably smaller than about 3. Non-limiting examples of potential insulation materials include Teflons®, such as polytetrafluoroethylene (PTFE), fluorinated ethylene propylene copolymer (FEP), perfluoroalkoxy (PFA), or ethylene and tetrafluoroethylene copolymer (ETFE, for example Tefzel®), or coatings other than Teflons®, such as polyetheretherketone plastics (PEEK™), parylene, certain ceramics, or polyethylene terpthalate (PET), or a range of other polymers. It should be emphasized that these materials are listed as non-limiting examples only, and any other suitable material with the appropriate dielectric properties could also be used as insulation. In some embodiments, the electrically insulating material forms a layer that extends substantially radially outwardly from the electrical conductor.

The heat shield shown in the embodiment in FIG. 3 can be used in other embodiments as well. For example, spacing apart the guiding element 173 and the ultrasound transducer 175 from the RF electrode 174 by the use of an intervening heat shield ensures that any temperature increase caused by the delivery of electrical energy to the target location only minimally influences the magnetic properties of the magnetic guiding element. Indeed, some materials, such as for example permanently magnetized materials, have a temperature over which they lose their magnetic properties. The heat shield can be used with any of the other embodiments described here as well.

Such a heat shield could be made out of a substantially thermally insulating material, for example, and non-limitingly, polytetrafluoroethylene (PTFE), which has a thermal conductivity of about 0.3 W/m-K. In this embodiment, the heat shield may have a thickness of at least about 0.025 mm. In other embodiments, the thickness of the heat shield may vary, depending on the thermal conductivity of the material being used. In some embodiments of the invention, the heat shield includes polytetrafluoroethylene (PTFE). The use of PTFE is advantageous as, in addition to having suitable thermal insulation properties, PTFE is also an electrically insulating material (having a dielectric strength of about 24 kV/mm) and, therefore, contributes to the prevention of arcing between the electrode and any metallic material that may be present in the guiding element. In alternate embodiments, other materials, such as for example, Zirconium Oxide, may be used for the heat shield.

In some embodiments of the invention, the guiding component(s) of either the integrated energy delivery apparatus or the magnetic RF guidewire include permanently magnetized components such as, for example a neodymium magnet, a platinum-cobalt magnet, or any other suitable heat-resistant magnets. A heat resistant magnet, for the purpose of this description, is defined as a magnet that has relatively low probabilities of being adversely affected in its magnetization by a delivery of electrical energy through the electrode. However, in alternative embodiments of the invention, each of the guiding components can include any other suitable magnetically responsive material such as, for example, a ferromagnetic, a paramagnetic, or a diamagnetic material.

In some embodiments of the invention, the electrical conductor of the body of the RF guidewire defines a conductor wider section and a conductor narrower section. The conductor narrower section is positioned distally relatively to the conductor wider section. The conductor wider section has a cross-sectional area that is substantially larger than the cross-sectional area of the conductor narrower section. The conductor narrower section increases the flexibility of the distal end section of the RF guidewire while the conductor wider section allows for maintaining a relatively large rigidity at the proximal end of the RF guidewire. This allows to relatively easily steer the conductor distal end while allowing to relatively easily manipulate the energy delivery apparatus into the body vasculature of the patient. In addition, having a conductor wider section of a relatively large cross-sectional area reduces ohmic losses when the electrical current is delivered to the RF electrode.

In some embodiments of the invention, the conductor wider and narrower section are substantially cylindrical and define respective conductor wider and narrower section outer diameters. Therefore, in these embodiments, the conductor wider section outer diameter is substantially larger than the conductor narrower section outer diameter. When the conductor material is Nitinol, a conductor narrower section having a conductor narrower section outer diameter of about 0.0027 inches or less has been found to be particularly well suited for use in relatively small body vessels.

In alternative embodiments of the invention, the electrical conductor is made more flexible substantially adjacent the conductor distal end than substantially adjacent the conductor proximal end in any other suitable manner such as, for example, by using different materials for manufacturing the conductor proximal and distal regions. It has been found that one suitable material for manufacturing the actual conductor is Nitinol. Indeed, Nitinol shows super-elastic properties and is therefore particularly suitable for applying relatively large deformations thereto in order to guide the energy delivery apparatus through relatively tortuous paths. Also, since the energy delivery apparatus typically creates channels inside biological tissues through radio frequency perforations, in some embodiments of the invention, the energy delivery apparatus typically does not need to be very rigid. In other embodiments, it is desirable that at least a substantial proximal section of the energy delivery apparatus have sufficient mechanical rigidity. Such rigidity or stiffness aids the use of the energy delivery apparatus as a rail to support and guide other therapeutic devices, such as catheters to the desired target location. Accordingly, a relatively stiff material, such as stainless steel can also be used as a substantial portion of the conductor.

It is desirable that preferably an insulation coating thickness of at least 0.002 inches, and still more preferably 0.003 inches is used as the insulation coating thickness. It is also preferable that the dielectric coating have a dielectric constant that is smaller than about 3, and more preferably smaller than about 2.5, and still more preferably smaller than about 2. For a 0.014″ (outer) diameter guidewire, this means that the conductor wire has a diameter of about 0.010 inches or smaller, and more preferably about 0.008 inches or smaller. As another example, in the case of a 0.018″ (outer) diameter guidewire, the conductor wire has a diameter of about 0.014 inches or smaller, and more preferably about 0.012 inches or smaller. In some applications it is desirable to use a wire conductor material that possesses a certain amount of mechanical stiffness. Thus, in the case of Nitinol, it is often desirable to use a wire conductor diameter of about 0.012 inches along the major proximal portion of the wire. Equivalently, if stainless steel is used as the wire conductor, it is desirable to use a wire conductor diameter of about 0.008 inches along the major proximal portion of the wire.

The above considerations can also be expressed in terms of ratios. For instance it is preferable that along the major proximal portion of the wire, the ratio of the insulation coating thickness to the wire conductor diameter is greater than about 0.18, and still more preferable that this ratio is greater than about 0.36, in the case of a 0.014″ (outer) diameter guidewire. In the case of a 0.018″ (outer) diameter guidewire, it is preferable that along the major proximal portion of the wire, the ratio of the insulation coating thickness to the wire conductor diameter is greater than about 0.13, and still more preferable that this ratio is greater than about 0.23.

In some embodiments of the invention, the energy delivery apparatus is used such that a channel is created at least partially through the occlusion. This channel may be created by delivering energy through the electrode and advancing the apparatus distal end into the occlusion simultaneously or after delivering energy. Alternatively or additionally, the channel could be enlarged by varying the magnetic field direction to make adjustments in the steering orientation of the energy delivery apparatus and ablating with each orientation change. This channel enlargement can be performed after an initial channel has been created by pulling back or by advancement of the energy delivery apparatus (either the magnetic RF guidewire or the integrated energy delivery apparatus), with steering adjustments throughout to enlarge the channel. Repeated passes of this process can also be performed to ensure that an adequately large channel is created.

It has been found that the claimed energy delivery apparatus is particularly well suited for creating channels in occlusions that are located at a bifurcation in the body vessel. Indeed, in prior art devices, the presence of the occlusion at the bifurcation typically pushes the apparatus distal end of prior art devices through the non-occluded branch of the body vessel, which therefore makes the creation of channels through the occlusion relatively difficult. By using the magnetic field, the apparatus distal end may be oriented such that the electrode remains substantially adjacent to the occlusion until at least a portion of a channel is created into the occlusion which allows the distal end of the energy delivery apparatus to be received within the occlusion, such that the energy delivery apparatus is guided away from the non-occluded branch.

In specific embodiments of the invention, the electrical conductor used for RF energy delivery is between about 40 centimeters and about 350 centimeters in length. In more specific embodiments of the invention, the electrical conductor is between about 65 centimeters and 265 centimeters in length. The electrode is typically less than about 4 millimeters in length.

In some embodiments, the heat shield 28 may be between about 0.05 cm and about 0.20 cm in length, and between 0.025 and about 0.05 cm in thickness. In one particular example, the heat shield material is about 0.1 cm in length, and about 0.035 cm in thickness.

It is appreciated that certain features of the invention, which are, for clarity, described in the context of separate embodiments, may also be provided in various combinations in a single embodiment. Conversely, various features of the invention, which are, for brevity, described in the context of a single embodiment, may also be provided separately or in any suitable subcombination.

Although the present invention has been described hereinabove by way of certain embodiments thereof, it can be modified, without departing from the subject invention as defined in the appended claims.

1. An occlusion crossing apparatus for creating an opening in occluded tissue at a target location with the use of intravascular ultrasound imaging, said occlusion crossing apparatus being usable in combination with a magnetic field, said occlusion crossing apparatus comprising:
an ultrasound imaging catheter with at least one ultrasound transducer disposed in its distal portion for transmission and reception of ultrasound energy;
said ultrasound transducers attached to leads that run through the length of the imaging catheter and connect at its proximal end to an ultrasound imaging system;
said ultrasound imaging catheter including a lumen through which a magnetically steered guidewire is passed and extends beyond the distal end of the imaging catheter;
at least one guiding element mounted to said guidewire, at least one of said guiding elements including a magnetically responsive material; and
whereby said guiding element in the portion of the guidewire extending beyond the distal end of the imaging catheter is capable of being steered in order to position said guidewire tip substantially adjacent to said target location by application of a suitable magnetic field.

2. The magnetically steered guidewire of claim 1, where the guidewire is a Radio Frequency guidewire comprising an electrical conductor, said guidewire also comprising an electrode in its distal portion for delivering Radio Frequency electrical energy at a target location in order to create a tissue opening at said target location, said electrode being electrically coupled to said electrical conductor.

3. The magnetically steered guidewire of claim 1, where the guidewire is mechanically pushed through the occlusion to create an opening at said target location.

4. The ultrasound imaging catheter of claim 1, where the catheter is a side-viewing catheter that provides signal data for reconstruction of an image of a radially disposed sector around the catheter by transmission of a radially disposed ultrasound beam from the at least one transducer.

5. The ultrasound imaging catheter of claim 1, where the catheter is an oblique-viewing catheter that provides signal data for reconstruction of an image of a obliquely disposed sector with both radial and longitudinal components with respect to the catheter, by transmission of an obliquely disposed ultrasound beam from the at least one transducer.

6. The ultrasound imaging catheter of claim 5, where the image data from the obliquely disposed sector is mapped into a circular image display by use of a suitable mapping function.

7. The circular image display of claim 6, where the circular image display includes a radial distance scale with distance markings indicating distance in front of the catheter.

8. The occlusion crossing apparatus of claim 1, where at least one of the ultrasound imaging catheter or the magnetically steered guidewire is remotely advanced by means of a remotely operated device advancer system.

9. The ultrasound imaging catheter of claim 1, where the catheter is rotated about its axis by means of a remotely operated device rotation system in order to sweep across multiple imaging sectors to acquire a larger imaging field of view.

10. The ultrasound imaging catheter of claim 1, where multiple ultrasound transducers in the distal portion of the catheter are operated to acquire image signal data in phased array form for reconstruction of a substantially complete circular field of view.

11. The ultrasound imaging catheter of claim 1, where the imaging catheter incorporates a metallic braid in the wall of the catheter.

12. The ultrasound imaging catheter of claim 1, where the imaging catheter incorporates at its distal tip at least one heat shield constructed from at least one material with low thermal conductivity.

13. The ultrasound imaging catheter of claim 1, where ultrasound transducers are incorporated in the form of a helically wound piezo film in the distal portion of the catheter.

14. An energy delivery apparatus for delivering electrical energy at a target location with the use of intravascular ultrasound imaging in order to create a tissue opening, said energy delivery apparatus being usable in combination with a magnetic field, said energy delivery apparatus comprising:
an ultrasound imaging catheter with at least one ultrasound transducer disposed in its distal portion for transmission and reception of ultrasound energy;
said ultrasound transducers attached to leads that run through the length of the imaging catheter and connect at its proximal end to an ultrasound imaging system;
said ultrasound imaging catheter including an electrode capable of delivering Radio Frequency electrical energy at a target location, said electrode disposed substantially circumferentially opposite said at least one ultrasound transducer, and said electrode connected through an electrical lead running through the catheter to a Radio Frequency generator through a suitable connector at the proximal end of the catheter;
at least one guiding element incorporated in said ultrasound imaging catheter, at least one of said guiding elements including a magnetically responsive material;
a flexible catheter shaft section proximal to said guiding element; and
whereby said guiding element is capable of steering the distal tip of the catheter in order to position said electrode substantially adjacent to said target location by application of a suitable magnetic field.

15. A method for opening occluded tissue at a target location with the use of an occlusion crossing apparatus together with intravascular ultrasound imaging, said occlusion crossing apparatus being usable in combination with a magnetic field generated by a magnetic navigation system, said method comprising the steps of:
(a) positioning near a target location in a body lumen an ultrasound imaging catheter with at least one ultrasound transducer disposed in its distal portion for transmission and reception of ultrasound energy;
(b) said ultrasound transducers attached to leads that run through the length of the imaging catheter and connect at its proximal end to an ultrasound imaging system;
(c) transmitting and receiving ultrasound signals with said ultrasound transducers and processing said received signals to reconstruct and display an image of local anatomy adjacent to the ultrasound imaging catheter;
(d) determining, from the reconstructed image, proximity of a boundary wall of the body lumen to the imaging catheter;
(e) using the determined proximity to suitably steer a magnetic guidewire by application of a magnetic field with said magnetic navigation system so as to orient the guidewire away from said boundary wall and positioning the guidewire at a target location;
(f) creating a tissue opening at said target location with the guidewire;
(g) advancing the guidewire into the created tissue opening;
(h) advancing the imaging catheter by following the guidewire into the tissue opening; and
repeating steps (a) through (h) in order to cross an occluded body lumen.

16. The method of claim 15, where the tissue opening at said target location is performed by delivering Radio Frequency electrical energy through an electrode at the distal tip of the guidewire.

17. The method of claim 15, where at least one of the ultrasound imaging catheter or the magnetic guidewire is remotely advanced by means of a remotely operated device advancer system.

18. The method of claim 15, where the ultrasound imaging catheter is rotated about its axis by means of a remotely operated device rotation system in order to sweep across multiple imaging sectors to acquire a larger imaging field of view.

19. The method of claim 15, where the step of signal transmission and reception comprises phased array operation of the image acquisition and reconstruction process in order to reconstruct a substantially complete circular field of view.

20. The method of claim 15, where the step of image reconstruction and display of image data from an obliquely disposed imaging sector is mapped into a circular image display by use of a suitable mapping function.

21. A medical device for magnetic navigation within a body lumen of a patient, the medical device comprising a proximal end and a distal end, a hollow lumen therebetween, a tip magnet, an ultrasound transducer mounted on a rotatable MEMS structure, said structure being rotatable with respect to a device long axis to provide a three-dimensional ultrasound image data set providing substantial coverage of a lumen wall.

22. The medical device of claim 21, wherein the ultrasound transducer is mounted on a rotatable ring at a distance proximally from the device distal end.

23. The medical device of claim 21, wherein the ultrasound transducer is mounted on a rotatable end cap, said cap comprising at least one surface for attaching the ultrasound transducer, said cap further comprising a hollow lumen permitting advancement of a further medical device within and beyond the distal end of the medical device of claim 1.

24. A method for opening an occluded vessel lumen with the use of an occlusion-crossing de-bulking imaging catheter apparatus together with intravascular ultrasound imaging, said occlusion crossing apparatus being usable in combination with a magnetic field generated by a magnetic navigation system, said method comprising the steps of:
positioning near a target location in a body lumen an ultrasound imaging catheter with at least one ultrasound transducer disposed in its distal portion for transmission and reception of ultrasound energy;
said ultrasound transducers attached to leads that run through the length of the imaging catheter and connect at its proximal end to an ultrasound imaging system;
transmitting and receiving ultrasound signals with said ultrasound transducers and processing said received signals to reconstruct and display an image of local anatomy adjacent to the ultrasound imaging catheter;
determining, from the reconstructed image, proximity of a boundary wall of the body lumen to the imaging catheter; and
using the determined proximity to suitably rotate the imaging catheter and apply RF ablation energy to de-bulk occlusive material in the vicinity of RF energy-delivery electrodes on the imaging catheter.

25. An occlusion-crossing de-bulking system comprising: (a) an ultrasound imaging catheter apparatus for de-bulking an occluded vessel in a patient, said occlusion crossing apparatus incorporating: (i) at least one electrode in its distal tip region for delivery of ablative RF energy, (ii) at least one ultrasound transducer disposed in its distal portion for transmission and reception of ultrasound energy, (iii) leads that run from the at least one transducer through the length of the imaging catheter and connect at its proximal end to an ultrasound imaging system, (iv) leads that run from the at least one electrode through the length of the imaging catheter to an RF energy delivery apparatus, (v) a lumen through the interior length of the imaging catheter for passage of a second minimal access device, and (b) a second minimal access device incorporating at least one magnetic element suitable for magnetic steering of the second device.

26. The de-bulking imaging catheter device of claim 25, where the imaging catheter further incorporates at least one magnetic element for magnetic steering of the imaging catheter.
